(19) United States
(12) Patent Application Publication
(10) Pub. No.: US 2026/0041838 A1
(43) Pub. Date: Feb. 12, 2026

(54) PORTABLE EVACUATION SYSTEM

(71) Applicant: BOARD OF REGENTS OF THE UNIVERSITY OF NEBRASKA, Lincoln, NE (US)

(72) Inventors: Riaz Ur Rehman Mohammed, Lincoln, NE (US); Benjamin S. TERRY, Lincoln, NE (US)

(21) Appl. No.: 19/100,866

(22) PCT Filed: Aug. 3, 2023
(86) PCT No.: PCT/US2023/029414

§ 371 Date: Feb. 3, 2025

Related U.S. Application Data
(60) Provisional application No. 63/370,273, filed on Aug. 3, 2022.

(51) Int. Cl. A61M 3/02; A61M 1/00

(52) U.S. Cl. CPC A61M 3/0295; A61M 1/60; A61M 1/66; A61M 1/87; A61M 2210/1067

(57) ABSTRACT

A portable evacuation system includes a pressure vessel configured to retain a vacuum chamber. The pressure vessel includes a valve port configured to receive a valve. The portable evacuation system also includes a catheter having an inlet side and an outlet side. The inlet side is configured to interface with the vacuum chamber via the valve port. The portable evacuation system also includes a balloon disposed about an outer surface of the catheter. The balloon is disposed proximate to the outlet side of the catheter.

### CROSS-REFERENCE TO RELATED APPLICATION(S)

The present application is the U.S. National Phase of PCT Patent Application Number PCT/US23/29414, filed on Aug. 3, 2023, which claims the benefit under 35 U.S.C. § 119 (e) of U.S. Provisional Application Ser. No. 63/370,273, filed Aug. 3, 2022, titled “PORTABLE EVACUATION SYSTEM”, all of which are incorporated herein by reference in their entirety.

### TECHNICAL FIELD

The present invention generally relates to suction devices for medical purposes, and more particularly to suction devices for in the anal canal.

### BACKGROUND

Mammals experience anodermal stretching due to routine defecation. Typically, rigorous treatment methods are used to mitigate medical issues that may worsen due to anodermal stretching, such as anal fissures and anal fistulas. An anal fissure (AF) is a linear or spindle-shaped tear in the epithelial lining of the anal canal (the anoderm). AFs usually occurs along the posterior midline. AFs result when the anoderm experiences some initial trauma like a hard stool and is damaged. Individuals suffering from AFs feel a sharp pain during defecation, along with instances of bleeding. Routine defecation causes re-stretching of the anoderm, re-widens the fissure, leading to a recurrent cycle of bleeding and pain. Injury and pain to the anoderm exacerbate hypertonicity which results in reduced blood flow and poor healing. The recurrent cycle of anoderm stretch and injury creates a positive feedback loop that deepens the wound and prolongs the healing process. Existing treatments for AFs focus on the symptomatic relief of pain and the promotion of wound healing. Treatment methods can include providing oral and/or topical medications.

Chronic anal fissures (CAFs) represent a significant problem in medicine, causing excruciating pain and perpetuating cycles of injury and impaired healing.

Current treatment modalities for chronic anal fissures (CAFs) suffer from limitations including side effects and low success rates, leading to the widespread use of invasive sphincterotomy as the standard treatment. However, sphincterotomy permanently damages the internal anal sphincter muscle, resulting in long-term fecal/flatus incontinence in a significant percentage of patients. Therefore, it would be advantageous to provide a device, system, and method that cures the shortcomings described above.

### SUMMARY

An evacuation device is described, in accordance with one or more embodiments of the present disclosure. The evacuation device includes a catheter. The catheter includes a first cylindrical tube. The first cylindrical tube defines a lumen. The lumen extends from a distal end of the first cylindrical tube to a proximal end of the first cylindrical tube. The catheter includes a balloon. The balloon is coupled around the first cylindrical tube. The balloon is disposed between the distal end of the first cylindrical tube and the proximal end of the first cylindrical tube. The balloon is configured to inflate and deflate. The evacuation device includes a pressure vessel defining a vacuum chamber. The evacuation device includes a two-port valve. The two-port valve couples the proximal end of the first cylindrical tube to the pressure vessel. The two-port valve comprises a closed position and an open position. The distal end of the first cylindrical tube is not in fluid communication with the vacuum chamber when the two-port valve is in the closed position. The distal end of the first cylindrical tube is in fluid communication with the vacuum chamber via the lumen and the two-port valve when the two-port valve is in the open position. The pressure vessel is configured to maintain a vacuum pressure less than atmospheric pressure when the two-port valve is in the closed position. The pressure vessel is configured to suction from the distal end of the first cylindrical tube via the vacuum pressure when the distal end of the first cylindrical tube is in fluid communication with the vacuum chamber.

In some embodiments, the pressure vessel is configured to suction feces from the distal end of the first cylindrical tube to the pressure vessel when the two-port valve is in the open position.

In some embodiments, the cap is coupled around the first cylindrical tube at the distal end of the first cylindrical tube.

In some embodiments, the first cylindrical tube defines a first hole and a second hole. The first hole is disposed between the distal end of the first cylindrical tube and the second hole. The catheter comprises a second cylindrical tube. The second cylindrical tube is inserted through the first hole and the second hole such that a portion of the second cylindrical tube is disposed in the lumen. The balloon is disposed above the first hole. A distal end of the second cylindrical tube is inserted into the balloon.

In some embodiments, the evacuation device includes an inflation control device. The inflation control device is coupled to the second cylindrical tube and configured to inflate the balloon by forcing a fluid into the balloon.

In some embodiments, the inflation control device is configured to control a backflow of fluid from the balloon.

In some embodiments, the inflation control device comprises at least one of a syringe or a compression bulb.

In some embodiments, at least one of the cap, the first cylindrical tube, and the second cylindrical tube comprises a silicon material.

In some embodiments, a diameter of the balloon is larger than a diameter of the cap when the balloon is inflated. The diameter of the balloon is smaller than the diameter of the cap when the balloon is deflated.

In some embodiments, the two-port valve is a ball valve.

In some embodiments, the evacuation device includes a check valve. The check valve is coupled to the pressure vessel and in fluid communication with the vacuum chamber.

In some embodiments, fluid is configured to be suctioned from the pressure vessel via the check valve to charge the pressure vessel with the vacuum pressure.

In some embodiments, the first cylindrical tube is a rigid cylindrical tube.

In some embodiments, the evacuation device is handheld.

An evacuation system is described, in accordance with one or more embodiments of the present disclosure. The evacuation system includes a pump. The evacuation system includes an evacuation device. The evacuation device includes a catheter. The catheter includes a first cylindrical tube. The first cylindrical tube defines a lumen. The lumen extends from a distal end of the first cylindrical tube to a proximal end of the first cylindrical tube. The catheter includes a balloon. The balloon is coupled around the first cylindrical tube. The balloon is disposed between the distal end of the first cylindrical tube and the proximal end of the first cylindrical tube. The balloon is configured to inflate and deflate. The evacuation device includes a pressure vessel defining a vacuum chamber. The evacuation device includes a two-port valve. The two-port valve couples the proximal end of the first cylindrical tube to the pressure vessel. The two-port valve comprises a closed position and an open position. The distal end of the first cylindrical tube is not in fluid communication with the vacuum chamber when the two-port valve is in the closed position; wherein the distal end of the first cylindrical tube is in fluid communication with the vacuum chamber via the lumen and the two-port valve when the two-port valve is in the open position. The pressure vessel is configured to maintain a vacuum pressure less than atmospheric pressure when the two-port valve is in the closed position. The pressure vessel is configured to suction from the distal end of the first cylindrical tube via the vacuum pressure when the distal end of the first cylindrical tube is in fluid communication with the vacuum chamber.

In some embodiments, the pump is fluidically coupled to the vacuum chamber. The pump is configured to remove fluid from the vacuum chamber to charge the vacuum chamber with the vacuum pressure.

In some embodiments, the evacuation system includes a topical agent applied to a cap of the catheter.

In some embodiments, the topical agent comprises at least one of a lubrication agent, a numbing agent, or a therapeutical agent.

A method is described, in accordance with one or more embodiments of the present disclosure. The method includes evacuating a vacuum chamber defined by a pressure vessel of an evacuation device. The evacuation device includes a catheter. The catheter includes a first cylindrical tube. The first cylindrical tube defines a lumen. The lumen extends from a distal end of the first cylindrical tube to a proximal end of the first cylindrical tube. The catheter includes a balloon. The balloon is coupled around the first cylindrical tube. The balloon is disposed between the distal end of the first cylindrical tube and the proximal end of the first cylindrical tube. The balloon is configured to inflate and deflate. The evacuation device includes the pressure vessel defining the vacuum chamber. The evacuation device includes a two-port valve. The two-port valve couples the proximal end of the first cylindrical tube to the pressure vessel. The two-port valve comprises a closed position and an open position. The distal end of the first cylindrical tube is not in fluid communication with the vacuum chamber when the two-port valve is in the closed position. The distal end of the first cylindrical tube is in fluid communication with the vacuum chamber via the lumen and the two-port valve when the two-port valve is in the open position. The pressure vessel is configured to maintain a vacuum pressure less than atmospheric pressure when the two-port valve is in the closed position. The pressure vessel is configured to suction from the distal end of the first cylindrical tube via the vacuum pressure when the distal end of the first cylindrical tube is in fluid communication with the vacuum chamber. The method includes inserting the distal end into an anal opening. The method includes inflating the balloon. The method includes opening the two-port valve causing the pressure vessel to suction feces from the distal end. The method includes closing the two-port valve. The method includes deflating the balloon. The method includes removing the distal end from the anal opening.

In some embodiments, the method includes applying a topical agent to a cap of the catheter.

### BRIEF DESCRIPTION OF THE DRAWINGS

The numerous advantages of the disclosure may be better understood by those skilled in the art by reference to the accompanying figures in which:

FIGS. 1A-1B depict an isometric view of an evacuation device with a balloon in a deflated state and a two-port valve in a closed position, in accordance with one or more embodiments of the present disclosure.

FIG. 1C depicts an isometric view of an evacuation device with a balloon in an inflated state and a two-port valve in a closed position, in accordance with one or more embodiments of the present disclosure.

FIG. 1D depicts an isometric view of an evacuation device with a balloon in an inflated state and a two-port valve in an open position for suctioning through a distal end of a catheter, in accordance with one or more embodiments of the present disclosure.

FIG. 1E depicts a cross-section view of an evacuation device with a balloon in an inflated state and a two-port valve in an open position for suctioning through a distal end of a catheter,

FIG. 1F depicts an exploded view of a catheter, in accordance with one or more embodiments of the present disclosure.

FIG. 2 depicts an evacuation system including an evacuation device, in accordance with one or more embodiments of the present disclosure.

FIG. 3 depicts a cross-section view of an evacuation device with a catheter inserted into an anal canal, in accordance with one or more embodiments of the present disclosure.

FIG. 4 depicts a flow diagram of a method, in accordance with one or more embodiments of the present disclosure.

### DETAILED DESCRIPTION OF THE INVENTION

The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure. Reference will now be made in detail to the subject matter disclosed, which is illustrated in the accompanying drawings.

Embodiments of the present disclosure are generally directed to a portable evacuation system. The portable evacuation system can be used to evacuate and/or extract fecal matter to mitigate defecation-related worsening of anal fissures and/or anal fistulas. The portable evacuation system reduces anoderm stretching during defecation. The reduction in anoderm stretching may lead to reduced pain during defecation and improved healing of anal fissures.

The portable evacuation system includes an evacuation device. The evacuation device includes a pressure vessel that retains a vacuum chamber. A catheter is connected to the vacuum chamber. The catheter can be inserted into an anal portion of a mammal. A two-port valve can be transitioned to an open position such that a distal portion of the catheter, i.e., the portion of the catheter inserted into the anus, causes a pressure differential. The resulting pressure differential can cause fecal matter to be transported to the vacuum chamber via the catheter.

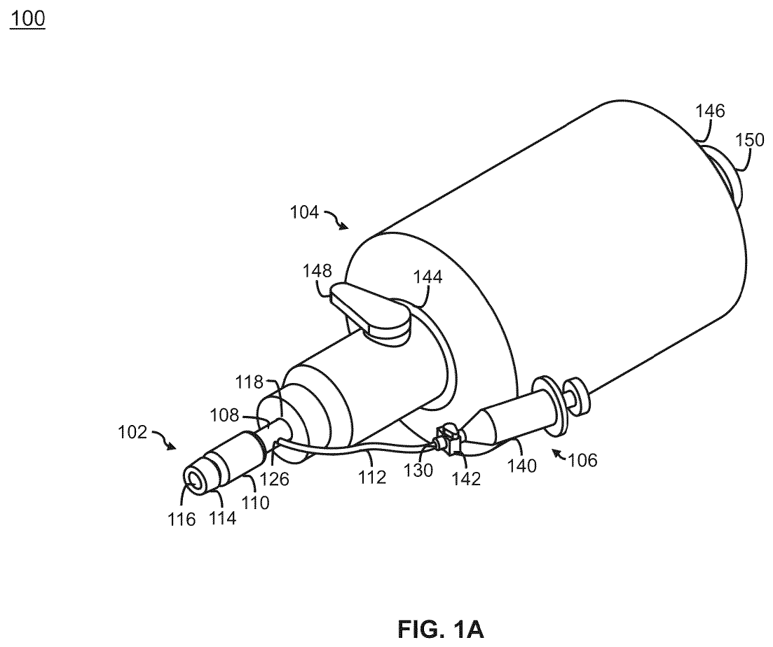

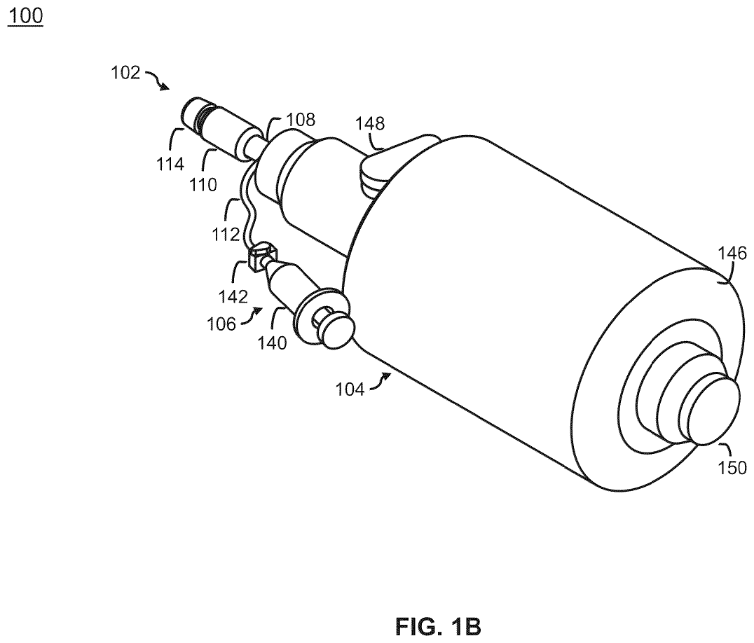

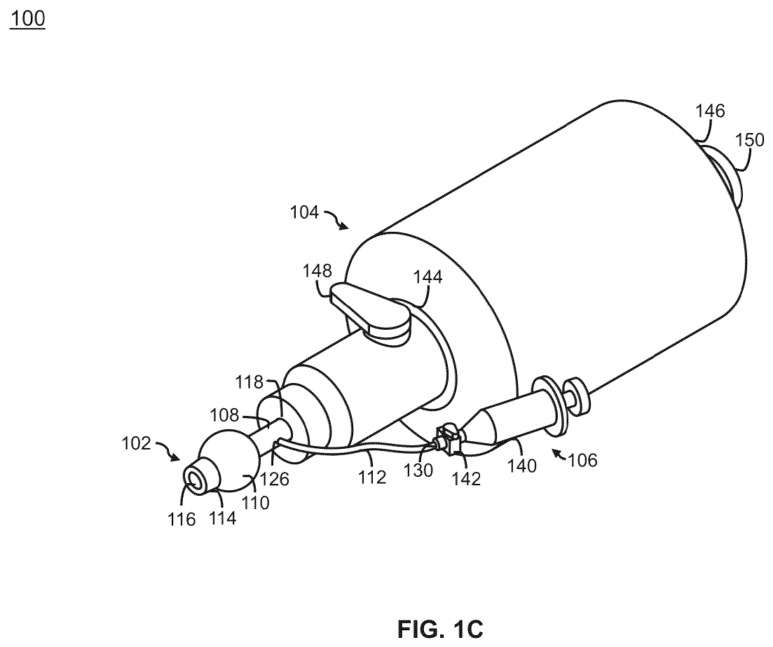

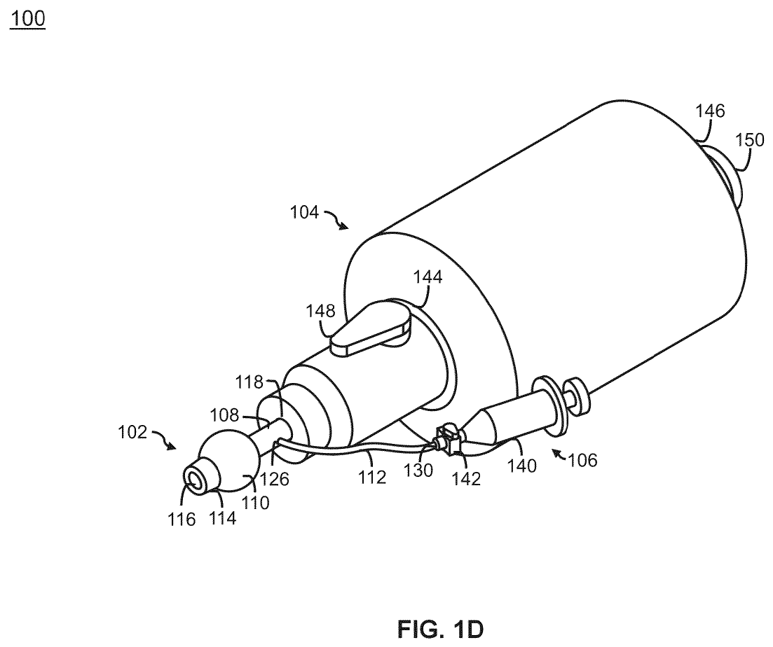

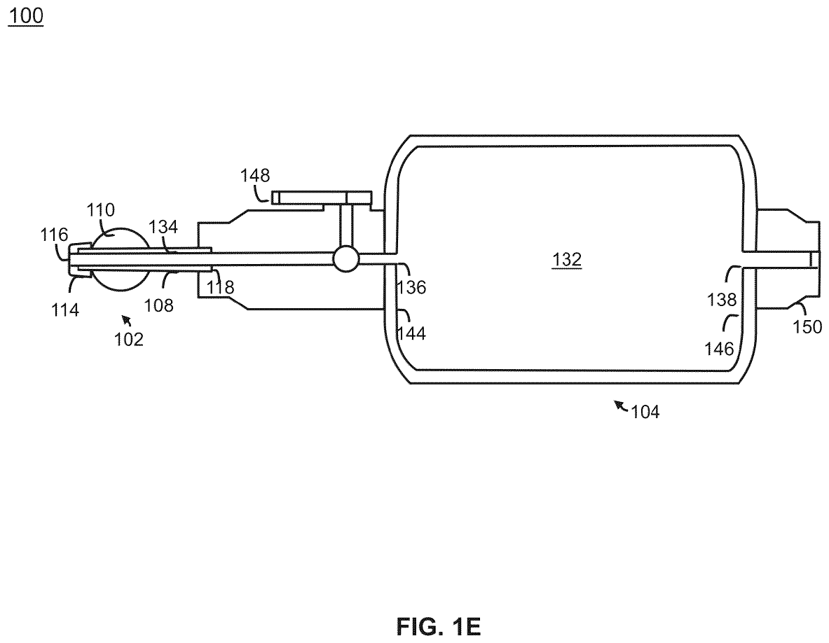

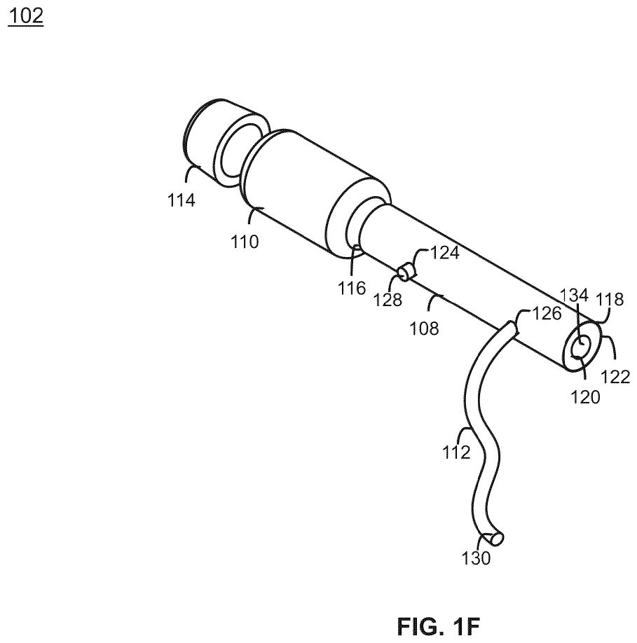

Referring now to FIGS. 1A-1F, an evacuation device 100 is described, in accordance with one or more embodiments of the present disclosure. The extraction device may also be referred to as a portable evacuation device. The evacuation device 100 includes one or more components, such as, but not limited to, a catheter 102, a pressure vessel 104, an inflation control device 106, two-port valve 148, check valve 150, and the like.

The evacuation device 100 includes the catheter 102. The catheter 102 may also be referred to as a rectal catheter, a rigid-wall rectal catheter, or the like. The catheter 102 includes one or more components, such as, but not limited to, a cylindrical tube 108, balloon 110, cylindrical tube 112, cap 114, and the like.

The catheter 102 includes the cylindrical tube 108. The cylindrical tube 108 may also be referred to as a first cylindrical tube, a fecal-transport tube, or the like. The cylindrical tube 108 includes a distal end 116, a proximal end 118, an inner diameter 120, an outer diameter 122. The inner diameter 120 may also be referred to as an inner surface. The outer diameter 122 may also be referred to as an outer surface. The difference between the inner diameter 120 and the outer diameter 122 is based on a wall thickness of the cylindrical tube 108.

The cylindrical tube 108 defines a lumen 134. For example, the inner diameter 120 of the cylindrical tube 108 defines the lumen 134. The lumen 134 extends from the distal end 116 of the cylindrical tube 108 to the proximal end 118 of the cylindrical tube 108. The lumen 134 includes a length. The length is selected to achieve a desired volumetric flow rate through the lumen 134 when the lumen 134 is subject to a vacuum pressure. The length impacts a pressure drop across the lumen 134 and therefore a flowrate through the lumen 134. The length must also be sufficiently long to reach a desired target location. In some embodiments, the length may be at least two inches. For example, the length may be selected to exceed a typical length of the anal canal from the anal opening to the pectinate line.

In some embodiments, the cylindrical tube 108 is a rigid cylindrical tube. In this regard, the cylindrical tube 108 may be formed of a rigid material. The cylindrical tube 108 being rigid causes the cylindrical tube 108 to prevent from collapsing when the lumen 134 of the cylindrical tube 108 is subject to vacuum pressure. The rigid cylindrical tube may also be referred to as a hard-walled tube, a non-flexible tube, or the like.

The cylindrical tube 108 defines a hole 124 and a hole 126. The holes may also be referred to as apertures. The hole 124 and the hole 126 are defined from the inner diameter 120 to the outer diameter 122. The hole 124 may also be referred to as a first hole. The hole 126 may also be referred to as a second hole. The hole 124 is defined at a first length from the distal end 116 of the cylindrical tube 108. The hole 126 is defined at a second length from the distal end 116 of the cylindrical tube 108. The hole 124 is disposed between the distal end 116 of the cylindrical tube 108 and the hole 126. Similarly, the hole 126 is disposed between the proximal end 118 of the cylindrical tube 108 and the hole 124.

The catheter 102 also includes the balloon 110. In this regard, the catheter 102 may be considered a balloon catheter or a balloon-type catheter. The balloon 110 is configured to inflate and deflate. The balloon 110 can be inflated to an inflated state and deflated to a deflated state. The balloon 110 is configured to hold a fluid. The fluid may include air, water, or the like. The fluid causes the balloon 110 to inflate. Backflow of the fluid from the balloon 110 causes the balloon to deflate. The balloon 110 is configured to hold a volume of the fluid. For example, the balloon 110 has a capacity to hold 32 mL of fluid, although this example is not intended to be limiting. In some embodiments, a diameter of the balloon 110 is larger than a diameter of the cap 114 when the balloon 110 is inflated. The diameter of the balloon 110 is smaller than the diameter of the cap 114 when the balloon is deflated. In an example implementation, the balloon 110 may comprise a silicone balloon.

The balloon 110 is coupled around the outer diameter 122 of the cylindrical tube 108. In this regard, the balloon 110 wraps around the cylindrical tube 108. The balloon 110 is disposed between the distal end 116 of the cylindrical tube 108 and the proximal end 118 of the cylindrical tube 108. The balloon 110 may be disposed over the outer surface of the cylindrical tube 108. The balloon 110 is attached to the cylindrical tube 108.

The catheter 102 includes the cylindrical tube 112. The cylindrical tube 112 may also be referred to as a second cylindrical tube 112, a balloon-inflation tube, or the like. The cylindrical tube 112 includes a distal end 128 and a proximal end 130. The distal end 128 may also be referred to as an outlet portion. The proximal end 130 may also be referred to as an inlet portion. The cylindrical tube 112 defines a lumen. For example, an inner diameter of the cylindrical tube 112 defines the lumen. The lumen extends from the distal end 128 of the cylindrical tube 112 to the proximal end 130 of the cylindrical tube 112.

The cylindrical tube 112 is received within the hole 124 and the hole 126. The cylindrical tube 112 is inserted through the hole 124 and the hole 126 such that a portion of the cylindrical tube 112 is disposed in the lumen 134 of the cylindrical tube 108. The distal end 128 of the cylindrical tube 112 protrudes from the hole 124. The proximal end 130 of the cylindrical tube 112 protrudes from the hole 126. The cylindrical tube 112 protrudes axially from the cylindrical tube 108 via the hole 124 and the hole 126.

In some embodiments, the cylindrical tube 112 is a soft-walled tube. In this regard, the cylindrical tube 112 may be formed of a material which is flexible. The cylindrical tube 112 being flexible allows the cylindrical tube 112 to bend for positioning the cylindrical tube 112. The soft-walled tube may also be referred to as a flexible tube or the like. In an example implementation, the cylindrical tube 112 comprises a silicone tube.

The balloon 110 is positioned above the distal end 128 of the cylindrical tube 112. The distal end 128 of the cylindrical tube 112 is inserted into the balloon 110. The cylindrical tube 112 provides a fluid pathway for inflating the balloon 110. In particular, the lumen of the cylindrical tube 112 provides the fluid pathway for inflating the balloon 110. The proximal end 130 of the cylindrical tube 112 provides fluid communication to the balloon 110 via the lumen of the cylindrical tube 112. The cylindrical tube 112 is then configured to provide fluid to the balloon 110 for inflation purposes.

The balloon 110 and the cylindrical tube 112 may be attached to the cylindrical tube 108 using any suitable technique. For example, the balloon 110 and the cylindrical tube 112 may be attached to the cylindrical tube 108 via a crimp ring, tape (e.g., polytetrafluoroethylene (PTFE) film tape), or the like.

The catheter 102 includes the cap 114. The cap 114 may also be referred to as a tip. The cap 114 is positioned over the distal end 116 of the cylindrical tube 108. The cap 114 is coupled around the outer diameter of the cylindrical tube 108 at the distal end 116 of the cylindrical tube 108. For example, the cap 114 may be coupled by an interference fit or the like (i.e., an inner diameter of the cap 114 is less than the outer diameter 122 of the cylindrical tube 108). The cap 114 permits suction through the distal end 116 of the cylindrical tube 108. For example, the cap 114 defines a hole. The hole of the cap 114 is aligned with the lumen 134 of the cylindrical tube 108. The hole of the cap 114 then permits suction through the distal end 116 of the cylindrical tube 108.

The cap 114 may include an outer diameter which is larger than an outer diameter of the cylindrical tube 108. The cap 114 may therefore reduce a pressure exerted by the cylindrical tube 108. For example, the reduced pressure may mitigate trauma to an orifice, such as an anus or rectum, during insertion and/or removal of the catheter 102. In some embodiments, the cap 114 is a soft-tipped cap. The soft-tipped cap may include any suitable material. For example, the cap 114 can comprise a silicone material. In some embodiments, one or more of the cap 114, the cylindrical tube 108, and the cylindrical tube 112 includes a silicon material.

The extraction device 100 also includes the inflation control device 106. The inflation control device 106 may also be referred to as an inflator. The inflation control device 106 is coupled to the cylindrical tube 112. For example, the inflation control device 106 is coupled to the proximal end 130 of the cylindrical tube 112.

The inflation control device 106 is in fluid communication with the balloon 110. The inflation control device 106 may force a fluid into the balloon 110 via the cylindrical tube 112. The fluid forced into the balloon 110 causes the balloon 110 to inflate. The inflation control device 106 may include a fluid pumping means. The fluid pumping means forces the fluid into the balloon 110. The fluid pumping means may be a manual fluid pumping means. For example, the inflation control device 106 may include a syringe 140, a compressible bulb, or the like.

In some embodiments, the inflation control device 106 may include one or more valves 142. The valve 142 controls or prevent the backflow of fluid from the balloon 110. The valve 142 may be actuatable to permit the backflow of fluid from the balloon 110. The backflow of fluid from the balloon 110 causes the balloon 110 to deflate. Although the inflation control device 106 is described as including the valve 142, this is not intended as a limitation of the present disclosure. It is contemplated that the syringe 140 may prevent the backflow of fluid when a plunger of the syringe 140 is depressed.

The evacuation device 100 also include the pressure vessel 104. The pressure vessel 104 may also be referred to as a housing. The pressure vessel 104 is a rigid body. The pressure vessel 104 may include a shape, such as, but not limited to, a cylindrical shape, a spherical shape, a rounded-rectangular shape, or the like. As depicted, the pressure vessel 104 is a spherical shape with rounded end caps, although this is not intended to be limiting.

The pressure vessel 104 defines a vacuum chamber 132. The vacuum chamber 132 may also be referred to as a feces collection chamber. The walls of the pressure vessel 104 defines the vacuum chamber 132. For example, an interior volume of the pressure vessel 104 defines the vacuum chamber 132. The vacuum chamber 132 is configured to maintain a constant volume when subject to a vacuum pressure. The pressure vessel 104 is configured to be charged with the vacuum pressure. Vacuum pressure refers to a pressure below atmospheric pressure. For example, vacuum pressure generally refers to a pressure between 0 and 1 standard atmosphere when measured at absolute pressure. The vacuum chamber 132 stores the vacuum pressure.

The pressure vessel 104 includes a port 136. The port 136 may also be referred to as a first port 136. In some embodiments, the port 136 is disposed on a side 144 of the pressure vessel 104. The port 136 interfaces with the two-port valve 148.

The evacuation device 100 includes the two-port valve 148. The two-port valve 148 includes a closed position and an open position. The two-port valve 148 prevents fluid flow in the closed position and permits fluid flow in the open position. In some embodiments, the two-port valve 148 is a ball valve.

The two-port valve 148 couples the proximal end 128 of the cylindrical tube 108 to the pressure vessel 104. The port 136 interfaces with the two-port valve 148. The two-port valve 148 is in fluid communication with and regulates the flow of fluid within the vacuum chamber 132. For example, the two-port valve 148 may positioned over the port 136. The distal end 116 of the cylindrical tube 108 is not in fluid communication with the vacuum chamber 132 when the two-port valve 148 is in the closed position. The vacuum chamber 132 maintains a relatively low-pressure environment when the two-port valve 148 is in a closed position. The pressure vessel 104 is configured to maintain a vacuum pressure less than atmospheric pressure when the two-port valve 148 is in the closed position.

The distal end 116 of the cylindrical tube 108 is in fluid communication with the vacuum chamber 132 via the lumen 134 and the two-port valve 148 when the two-port valve 148 is in the open position. The pressure vessel 104 is configured to suction from the distal end 116 of the cylindrical tube 108 via the vacuum pressure when the distal end 116 of the cylindrical tube 108 is in fluid communication with the vacuum chamber 132. The suction is induced by a pressure differential across the catheter 102. In this regard, a transfer of fluid is permitted from a relatively higher-pressure environment outside the distal end 116 to the low-pressure environment within the pressure vessel 104 when the two-port valve 148 is in an open position. For example, the pressure vessel 104 is configured to suction feces from the distal end 116 of the cylindrical tube 108 to the pressure vessel 104 when the two-port valve 148 is in the open position.

In some embodiments, the pressure vessel 104 includes a port 138. The port 138 may also be referred to as a charging port, a second port, or the like. The port 138 may be used to charge the vacuum chamber 132 with the vacuum pressure. The port 138 is configured to interface with a vacuum pump to create at least a partial vacuum within the vacuum chamber 132 of the pressure vessel 104. For example, the port 138 can be connected to a suction pump to allow the suction pump to remove fluid, such as gas molecules, from the vacuum chamber 132. Removing the fluid results in at least a partial vacuum relative to atmospheric pressure thereby charging the vacuum chamber 132 with the vacuum pressure. In some embodiments, the port 136 is disposed on a side 146 of the pressure vessel 104. The side 146 of the pressure vessel 104 may be opposite to the side 144 of the pressure vessel 104.

In some embodiments, the evacuation device 100 includes the check valve 150. The check valve 150 is coupled to the pressure vessel 104 and in fluid communication with the vacuum chamber 132. For example, the check valve 150 interfaces with the vacuum chamber 132 via the port 138. The check valve 150 may prevent the flow of fluid to the vacuum chamber 132 and permit the flow of fluid from the vacuum chamber 132. In this regard, the fluid may be suctioned from the vacuum chamber 132 via the check valve 150 to charge the vacuum chamber 132 with the vacuum pressure.

Although the pressure vessel 104 is described as including the port 138 used to charge the vacuum chamber 132 and the evacuation device 100 is described as including the check valve 150, this is not intended as a limitation of the present disclosure. It is contemplated that the port 136 may perform the functions of the port 136 and the two-port valve 148 may perform the functions of the check valve 150. For example, the port 136 and the two-port valve 148 may be used to charge the vacuum chamber 132 with the vacuum pressure. Removing the check valve 150 may simplify a construction of the evacuation device 100 and prevent a flow of fecal matter from the vacuum chamber 132.

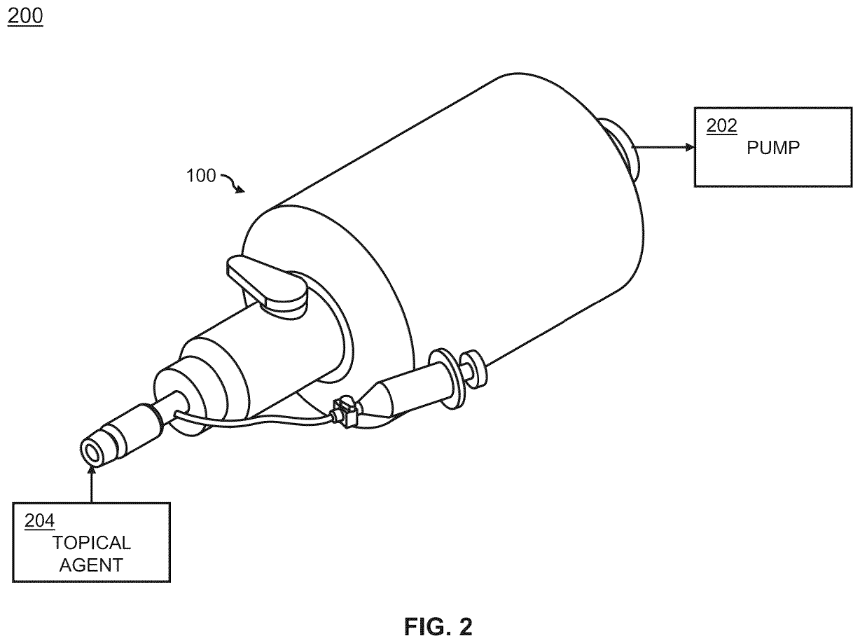

Referring now to FIG. 2, an evacuation system 200 is described, in accordance with one or more embodiments of the present disclosure. The evacuation system 200 may also be referred to as a portable evacuation system 200. The evacuation system 200 includes one or more components, such as, but not limited to, the evacuation device 100, pump 202, topical agent 204, and the like.

The evacuation system 200 includes the pump 202. The pump 202 may also be referred to as a vacuum pump, a non-disposable pump or the like. The pump 202 is fluidically coupled to the vacuum chamber 132. The pump 202 is configured to remove fluid from the vacuum chamber 132 to charge the vacuum chamber 132 with the vacuum pressure. In some embodiments, the pump 202 is coupled to the check valve 150. In some embodiments, the pump 202 is coupled to the two-port valve 148. The pump 202 is used to charge the vacuum chamber 132 during the day of use. The pump 202 may then be decoupled from the evacuation device 100, enabling the evacuation device 100 to be portable.

In some embodiments, the evacuation device 100 includes a vacuum gauge (not depicted). In some embodiments, the pump 202 includes the vacuum gauge. The vacuum gauge may provide a pressure reading of the vacuum chamber 132.

The evacuation system 200 includes the topical agent 204. The topical agent 204 is applied to the cap 114. The topical agent may include any number of topical agents which are compatible with insertion into the anus. For example, the topical agent can comprise a lubrication agent, a numbing agent, and/or a therapeutical agent.

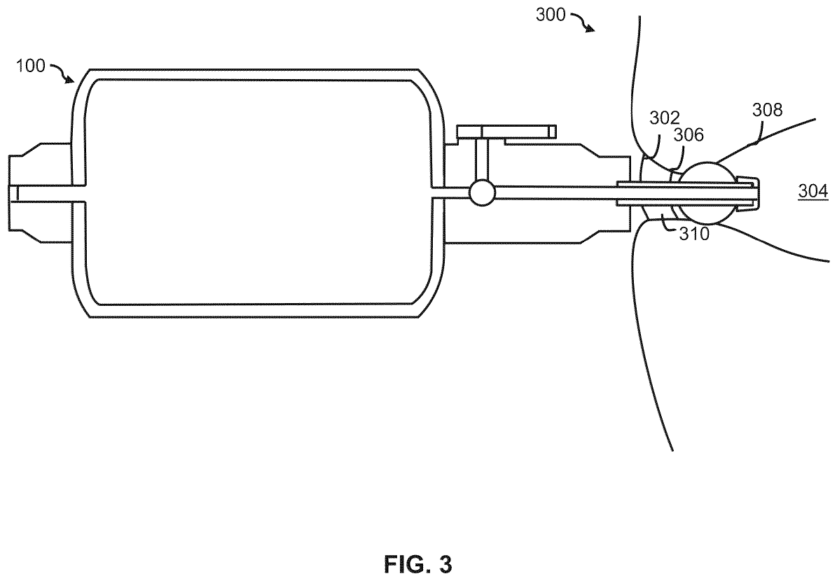

Referring now to FIG. 3, an example usage of the evacuation device 100 is described, in accordance with one or more embodiments of the present disclosure. The evacuation device 100 may be used within a human 300. The human 300 includes an anal opening 302, anal canal 304, pectinate line 306, anal sphincter 308, and anoderm 310. The pectinate line 306 is disposed inwards of the anal opening 302. The anal sphincter 308 is a tissue defining the anal canal 304. The anoderm 310 is a type of skin tissue disposed between the anal opening 302 and the pectinate line 306. The evacuation device 100 prevents repeated stretching of the anoderm 310 during defecation. Preventing the repeated stretching of the anoderm 310 may reduce pain, injury, and hypertonicity cycle and/or promote healing.

The distal end 116 and/or the balloon 110 are disposed at or inwards of the pectinate line 306. For example, the balloon 110 may be disposed approximately two inches above the anal opening 302.

The balloon 110 is in an inflated state, causing the balloon 110 to press against the anal sphincter 308. The balloon 110 exerts an outward force on the anal sphincter 308, holding the catheter 102 in place. The balloon 110 occludes the anal canal 304 in the inflated state. The diameter of the balloon 110 when the balloon is inflated is sufficiently large to occlude the anal canal 304. The occlusion of the anal canal 304 prevents flow of feces around the catheter 102. The balloon 110 also prevents aspiration of the anal sphincter 308 into the lumen 134 of the catheter 102.

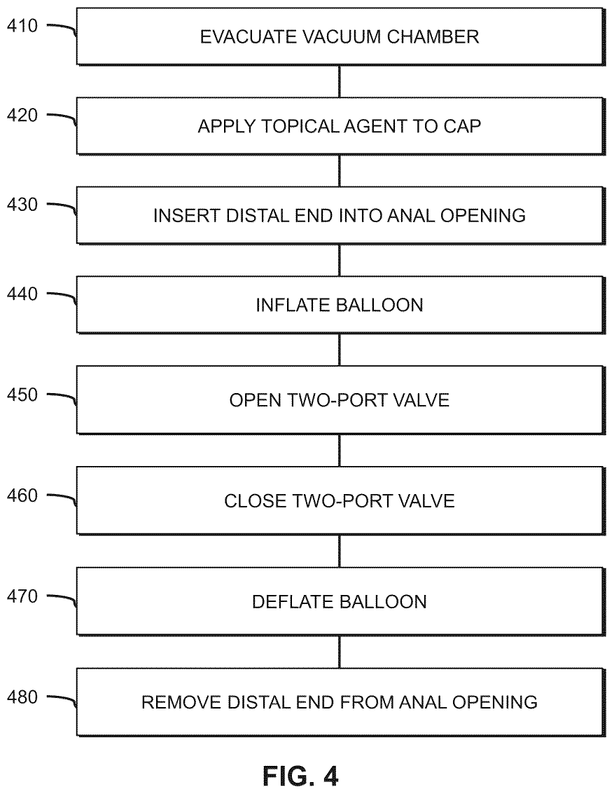

Referring now to FIG. 4, a flow diagram of a method 400 is described, in accordance with one or more embodiments of the present disclosure. The embodiments and enabling technologies described previously herein in the context of the evacuation device 100 and the evacuation system 200 should be interpreted to extend to method 400. For example, the method 400 may use the evacuation device 100 and the evacuation system 200 to perform any of the various steps of the method.

In a step 410, the vacuum chamber 132 of the evacuation device 100 is evacuated. The vacuum chamber 132 of the evacuation device 100 is evacuated by the pump 202. In this regard, the vacuum chamber 132 is pre-evacuated before insertion of the catheter 102 into the anal canal 304.

In a step 420, the topical agent 204 is applied to the cap 114 of the evacuation device 100. The cap 114 is lubricated with the topical agent 204 to ease insertion. The cap 114 with the topical agent 204 prevents trauma to the anal sphincter 308 from the catheter 102 edges during insertion.

In a step 430, the distal end 116 of the cylindrical tube 108 is inserted into the anal opening 302. The distal end 116 is inserted until the distal end 116 and/or the balloon 110 is disposed at or inwards of the pectinate line 306. For example, the balloon 110 may be actuated approximately two inches above the anal opening 302.

In a step 440, the balloon 110 is inflated. The inflation control device 106 inflates the balloon 110. The balloon 110 is inflated until the balloon 110 presses against the anal sphincter 308. The step 440 may optionally include closing the valve 142 in response to inflating the balloon. The valve 142 is closed to keep the balloon 110 inflated. The valve 142 keeps the balloon 110 inflated while the valve 142 is closed.

In a step 450, the two-port valve 148 is opened. The two-port valve 148 can be transitioned from the closed position to the open position. When the two-port valve 148 is positioned in the open position, the anal environment is subjected to a relatively lower pressure, which can cause the evacuation and/or extraction of fecal matter from the subject. For example, the feces can be transported to the vacuum chamber 132 via the catheter 102. The two-port valve 148 is opened to suction feces from the anal canal 304 into the vacuum chamber 132. The feces may include, but is not limited to, type-4 human feces (i.e., Bristol type-4 human feces). In some embodiments, the evacuation device 100 may extract 200-250 g of synthetic feces at a flowrate of 32 mL/s.

In a step 460, the two-port valve 148 is closed. The two-port valve 148 is closed to prevent backflow of the feces from the vacuum chamber 132 to the anal canal 304. The step 460 may optionally include opening the valve 142. The valve 142 may be opened in response to closing the two-port valve 148. The valve 142 is opened to enable fluid to backflow from the balloon 110.

In a step 470, the balloon 110 is deflated. The inflation control device 106 deflates the balloon 110. Backflow of fluid from the balloon 110 to the inflation control device 106 causes the balloon 110 to deflate.

In a step 480, the distal end 116 of the cylindrical tube 108 is removed from the anal opening 302. In this regard, the catheter 102 may then be removed from the anal opening 302 in response to deflating the balloon 110. An operator of the evacuation device 100 can then dispose of the pressure vessel 104 after use.

Referring generally again to FIGS. 1A-4, in some embodiments, the evacuation device 100 is disposable or single-use. For example, the two-port valve 148 is may be made of a material. The material may be a plastic material or the like. The evacuation device 100 may be discarded post-use in public use areas without necessitating a specialized disposal process.

In some embodiments, the evacuation device 100 is dimensioned to fit within a carrier bag. The evacuation device 100 is a disposable feces extraction unit that is small enough for portability and discreet use in public restrooms. The evacuation device 100 is lightweight and compact, able to be conveniently carried in a small bag or purse. The evacuation device 100 may then be easily transported to public bathrooms.

In some embodiments, the evacuation device 100 is handheld. The evacuation device 100 is sized to be held in one hand. The evacuation device 100 is designed to be operated while being held in one hand. The evacuation device 100 may allow for one-handed insertion. A second hand may then perform the activation of the two-port valve.

In some embodiments, The evacuation device 100 is vacuum-based and purely mechanical. In this regard, the evacuation device 100 does not include any electrical components.

In some embodiments, the catheter 102 may be detached from the two-port valve 148. The catheter 102 may then be cleaned and reused for repeated for repeated use.

It is further contemplated that each of the embodiments of the method described above may include any other step(s) of any other method(s) described herein. In addition, each of the embodiments of the method described above may be performed by any of the systems described herein.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, operations, devices, and objects should not be taken as limiting.

As used herein, directional terms such as “top,” “bottom,” “over,” “under,” “upper,” “upward,” “lower,” “down,” and “downward” are intended to provide relative positions for purposes of description, and are not intended to designate an absolute frame of reference. Various modifications to the described embodiments will be apparent to those with skill in the art, and the general principles defined herein may be applied to other embodiments

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mixable and/or physically interacting components and/or wirelessly interactable and/or wirelessly interacting components and/or logically interacting and/or logically interactable components.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. An evacuation device comprising:
a catheter comprising:
a first cylindrical tube; wherein the first cylindrical tube defines a lumen; wherein the lumen extends from a distal end of the first cylindrical tube to a proximal end of the first cylindrical tube; and
a balloon; wherein the balloon is coupled around the first cylindrical tube; wherein the balloon is disposed between the distal end of the first cylindrical tube and the proximal end of the first cylindrical tube; wherein the balloon is configured to inflate and deflate;
a pressure vessel defining a vacuum chamber; and
a two-port valve; wherein the two-port valve couples the proximal end of the first cylindrical tube to the pressure vessel;
wherein the two-port valve comprises a closed position and an open position; wherein the distal end of the first cylindrical tube is not in fluid communication with the vacuum chamber when the two-port valve is in the closed position; wherein the distal end of the first cylindrical tube is in fluid communication with the vacuum chamber via the lumen and the two-port valve when the two-port valve is in the open position;
wherein the pressure vessel is configured to maintain a vacuum pressure less than atmospheric pressure when the two-port valve is in the closed position; wherein the pressure vessel is configured to suction from the distal end of the first cylindrical tube via the vacuum pressure when the distal end of the first cylindrical tube is in fluid communication with the vacuum chamber.

2. The evacuation device of claim 1, wherein the pressure vessel is configured to suction feces from the distal end of the first cylindrical tube to the pressure vessel when the two-port valve is in the open position.

3. The evacuation device of claim 1, the catheter comprising a cap; wherein the cap is coupled around the first cylindrical tube at the distal end of the first cylindrical tube.

4. The evacuation device of claim 3, wherein the first cylindrical tube defines a first hole and a second hole; wherein the first hole is disposed between the distal end of the first cylindrical tube and the second hole;
wherein the catheter comprises a second cylindrical tube; wherein the second cylindrical tube is inserted through the first hole and the second hole such that a portion of the second cylindrical tube is disposed in the lumen;
wherein the balloon is disposed above the first hole; wherein a distal end of the second cylindrical tube is inserted into the balloon.

5. The evacuation device of claim 4, comprising an inflation control device;
wherein the inflation control device is coupled to the second cylindrical tube and configured to inflate the balloon by forcing a fluid into the balloon.

6. The evacuation device of claim 5, wherein the inflation control device is configured to control a backflow of fluid from the balloon.

7. The evacuation device of claim 5, wherein the inflation control device comprises at least one of a syringe or a compression bulb.

8. The evacuation device of claim 4, wherein at least one of the cap, the first cylindrical tube, and the second cylindrical tube comprises a silicon material.

9. The evacuation device of claim 4, wherein a diameter of the balloon is larger than a diameter of the cap when the balloon is inflated; wherein the diameter of the balloon is smaller than the diameter of the cap when the balloon is deflated.

10. The evacuation device of claim 1, wherein the two-port valve is a ball valve.

11. The evacuation device of claim 1, comprising a check valve; wherein the check valve is coupled to the pressure vessel and in fluid communication with the vacuum chamber.

12. The evacuation device of claim 11, wherein fluid is configured to be suctioned from the pressure vessel via the check valve to charge the pressure vessel with the vacuum pressure.

13. The evacuation device of claim 1, wherein the first cylindrical tube is a rigid cylindrical tube.

14. The evacuation device of claim 1, wherein the evacuation device is handheld.

15. An evacuation system comprising:
a pump;
an evacuation device comprising:
a catheter comprising:
a first cylindrical tube; wherein the first cylindrical tube defines a lumen; wherein the lumen extends from a distal end of the first cylindrical tube to a proximal end of the first cylindrical tube; and
a balloon; wherein the balloon is coupled around the first cylindrical tube; wherein the balloon is disposed between the distal end of the first cylindrical tube and the proximal end of the first cylindrical tube; wherein the balloon is configured to inflate and deflate;
a pressure vessel defining a vacuum chamber; and
a two-port valve; wherein the two-port valve couples the proximal end of the first cylindrical tube to the pressure vessel;
wherein the two-port valve comprises a closed position and an open position; wherein the distal end of the first cylindrical tube is not in fluid communication with the vacuum chamber when the two-port valve is in the closed position; wherein the distal end of the first cylindrical tube is in fluid communication with the vacuum chamber via the lumen and the two-port valve when the two-port valve is in the open position;
wherein the pressure vessel is configured to maintain a vacuum pressure less than atmospheric pressure when the two-port valve is in the closed position; wherein the pressure vessel is configured to suction from the distal end of the first cylindrical tube via the vacuum pressure when the distal end of the first cylindrical tube is in fluid communication with the vacuum chamber.

16. The evacuation system of claim 15, wherein the pump is fluidically coupled to the vacuum chamber; wherein the pump is configured to remove fluid from the vacuum chamber to charge the vacuum chamber with the vacuum pressure.

17. The evacuation system of claim 15, comprising a topical agent applied to a cap of the catheter.

18. The evacuation system of claim 17, wherein the topical agent comprises at least one of a lubrication agent, a numbing agent, or a therapeutical agent.

19. A method comprising:
evacuating a vacuum chamber defined by a pressure vessel of an evacuation device; the evacuation device comprising:
a catheter comprising:
a first cylindrical tube; wherein the first cylindrical tube defines a lumen; wherein the lumen extends from a distal end of the first cylindrical tube to a proximal end of the first cylindrical tube; and
a balloon; wherein the balloon is coupled around the first cylindrical tube; wherein the balloon is disposed between the distal end of the first cylindrical tube and the proximal end of the first cylindrical tube; wherein the balloon is configured to inflate and deflate;
the pressure vessel defining the vacuum chamber; and
a two-port valve; wherein the two-port valve couples the proximal end of the first cylindrical tube to the pressure vessel;
wherein the two-port valve comprises a closed position and an open position; wherein the distal end of the first cylindrical tube is not in fluid communication with the vacuum chamber when the two-port valve is in the closed position; wherein the distal end of the first cylindrical tube is in fluid communication with the vacuum chamber via the lumen and the two-port valve when the two-port valve is in the open position;
wherein the pressure vessel is configured to maintain a vacuum pressure less than atmospheric pressure when the two-port valve is in the closed position; wherein the pressure vessel is configured to suction from the distal end of the first cylindrical tube via the vacuum pressure when the distal end of the first cylindrical tube is in fluid communication with the vacuum chamber;
inserting the distal end into an anal opening;
inflating the balloon;
opening the two-port valve causing the pressure vessel to suction feces from the distal end;
closing the two-port valve;
deflating the balloon; and
removing the distal end from the anal opening.

20. The method of claim 19, comprising applying a topical agent to a cap of the catheter.
